(19) United States
(12) Patent Application Publication
(10) Pub. No.: US 2003/0148291 A1
(43) Pub. Date: Aug. 7, 2003

(54) Method of immobilizing biologically active molecules for assay purposes in a microfluidic format

(76) Inventor: Karla Robotti, Mountain View, CA (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(21) Appl. No.: 10/072,525

(22) Filed: Feb. 5, 2002

(51) Int. Cl. C12Q 1/68; G01N 33/53; G01N 33/542; G01N 33/552

(52) U.S. Cl. 435/6; 435/7.9; 436/527

(57) ABSTRACT

The invention provides biological molecules entrapped within a sol-gel matrix and incorporated into a microanalytical device for high throughput screening of samples. The pore sizes of the matrix may be chosen to match the size of the entrapped biological molecule or to correspond in size with the sample molecules to be analyzed. The sol-gel may be formed into structures that can be incorporated into or onto the microanalytical devices as microcolumns, microchannels, and microarrays. The sol-gel may incorporate substituted silanes and thereby provide a hydrophobic or hydrophilic surface, thereby providing the potential for use in microchromatography, microelectrophoresis or combinations thereof on the microanalytical device. A preferred detection method of samples is mass spectrometry.

### FIELD OF THE INVENTION

This invention relates generally to a method for immobilizing biomolecules without affecting their biological function. More specifically, the invention relates to immobilization of biomolecules in porous, inorganic matrices, and to methods of using the immobilized biomolecules in various contexts. The invention additionally relates to microfluidic systems wherein the immobilized biomolecules are contained within microchannels, microcolumns, or the like, for example, for performing high throughput screening in a microfluidic format.

### BACKGROUND

Assays performed in biological settings are often part of a sequential process involving sample preparation, binding or activity assay, clean up, separation and eventual detection. In the quest for new drug leads, the pharmaceutical industry is saddled with huge numbers of assays to be completed in as short a time as possible. High throughput screening (HTS) of small molecule drug candidates is required in order to identify these new drug leads. Further, there is a need for HTS methods that utilize a micro device format, as the microfluidic format has the advantage over conventional assays of requiring only small amounts of reagents and solvents and allowing for fast performance of the assays. High throughput screening in the microfluidic format also results in less human handling of materials and the capacity for unattended processing. To develop high throughput screening methods, the assays must be placed “on-line” or on-device, for example, by immobilizing the biomolecules (antibodies, enzymes, receptor proteins, and the like) in a manner that still retains their biological function. Prior ways of binding biomolecules include the following: a) physical adsorption and b) covalent binding. Adsorption of molecules often leads to leaching problems and a need to replenish the molecule in a dynamic manner. Covalent binding, which overcomes the leaching problem, requires suitable chemical moieties on the biomolecule to which one can chemically react the biomolecule and attach it to the device. Even when these moieties exist, there is a possibility that the binding may modify or interfere with the active site of the biomolecule rendering it biologically inactive after attachment or causing it to have reduced or modified activity).

Molecules and cellular structures have previously been immobilized by entrapment; however, the entrapment was usually accomplished using organic polymers such as polyvinyl alcohol or polyacrylic acid as the trapping medium. Such organic polymer based entrapment often resulted in weaker gel networks that broke apart and leached the biomolecule.

Inorganic silicates (silicate glass matrices) have been used more recently to entrap enzymes, as described in Narang et al. (1994), “Glucose Biosensor Based on a Sol-Gel Derived Platform,” Anal. Chem. 66(19):3139-3144; Braun et al. (1990), “Biochemically Active Sol-Gel Glasses: The Trapping of Enzymes,” Materials Lett. 10(1,2): 1-5; and Johnson et al. (1971), “On the Use of Polymerizing Silica Gel Systems for the Immobilization of Trypsin,” J. Colloid and Interface Sci. 37(3):557-563. The biomolecule is entrapped by the growth or polymerization of a porous silicate structure around the biomolecule. However, the references cited above report that enzymes entrapped within the silicate matrix do not perform as well as soluble enzymes, such as are used in a typical enzyme digestor. Although the matrix is quite porous, the pore sizes in the glass matrix are limiting if the reactant of choice is also as large as the enzyme that was trapped. The pore size is critical in a digest chamber, since large proteins to be digested will not be able to enter the sol-gel matrix.

In addition to the references mentioned above, the following relate to one or more aspects of the present invention, and reference may be had thereto for background information not explicitly included herein.

U.S. Pat. No. 5,200,334 to Dunn reports that enzymes may be entrapped in sol-gels, wherein a metal alkoxide is mixed with water and exposed to ultrasonic energy at a defined pH to form a single phase solution which is then buffered to a pH between about 5 and 7. The buffered solution is then mixed with the active biological material and the resultant gel is aged and dried. Dunn reports that the dried product is a transparent porous glass with substantially all of the added active biological material entrapped therein, and that the biological material retained a high level of activity.

U.S. Pat. No. 5,300,564 to Avnir reports a proposed method of obtaining a chemical interaction between at least one reagent trapped in sol-gel glass by doping it with the reagent, and diffusible solutes or components in an adjacent liquid or gas phase, whrerein the reagents, the solutes or the components can be any organic or inorganic compounds or materials of biological origin including enzymes. The doped sol-gel glass in various forms may be useful as analytical test, chromatographic medium, sensor, catalyst or biocatalyst, electrode or enzyme electrode, or other detection device.

U.S. Pat. No. 6,180,378 to Shen reports immobilized bioactive protein compositions can be prepared containing a bioactive protein such as an enzyme intercalated into galleries of a phyllosilicate, and a crosslinking compound crosslinking the phyllosilicate and the bioactive protein. The phyllosilicate may contain sodium or alkylammonium ions and be montmorillonite. The protein may be lipoxygenase, and crosslinking compounds include tetramethyl orthosilicate, tetraethoxy silicate, propyltrimethoxy silicate, polydimethyl orthosilicate, and methyltrimethoxy silicate. The composition is prepared by delaminating a sodium-saturated phyllosilicate, mixing a bioactive protein with the delaminated phyllosilicate and crosslinking with a crosslinking compound. After crosslinking, the composition may be vacuum dried and ground. The composition can also be prepared by delaminating a montmorillonite, saturating the delaminated montmorillonite with sodium ions, mixing the resultant montmorillonite with an enzyme, adding tetramethyl orthosilicate, allowing crosslinking, and drying. Activities of up to 170% of free protein are achieved using the immobilized bioactive protein compositions, and the compositions retain up to 98% original activity after being stored at room temperature for two weeks.

U.S. Pat. No. 6,303,290 to Liu relates to a process for the encapsulation of biologically important proteins into transparent, porous silica matrices by an alcohol-free, aqueous, colloidal sol-gel process, and to the biological materials entrapped thereby. Conformation, and hence activity of the biomaterial, is reported to be successfully retained after encapsulation as demonstrated by optical characterization of the molecules, even after long-term storage. The retained conformation of the biomaterial is reported to be strongly correlated to both the rate of gelation and the subsequent drying speed of the encapsulating matrix. Moreover, in accordance with this process, gelation is accelerated by the use of a higher colloidal solid concentration and a lower synthesis pH than conventional methods, thereby enhancing structural stability and retained conformation of the biomaterials. As reported by Liu, the gel shrinks as it dries, and it hardens further into an inorganic matrix with a pore size typically in the range of 2-5 nm. According to Liu, the matrix pores allow the diffusion of reactant molecules and their reaction with the entrapped biomolecules, and the biomolecule-containing inorganic monolith materials possess an ability to detect other molecules, and can thus be used as sensors for optical, electrical, mechanical or chemical signals.

As reported in Altstein et al. (2001), “Immunochemical Approaches for Purification and Detection of TNT Traces by Antibodies Entrapped in a Sol-Gel Matrix,” Anal. Chem. 73(11):2461-2467, the activity of entrapped biomolecules may be enhanced in the sol-gel, relative to unentrapped biomolecules and may be due to the structural stability provided by the sol-gel matrix to the entrapped biomolecule. The enhanced stability is seen in the resistance of the entrapped biomolecules (IgGs) to denaturation by solvents.

In sample analysis instrumentation, smaller dimensions generally result in improved performance characteristics and at the same time result in reduced production and analysis costs. Miniaturized separation systems, for example, provide more effective system design, result in lower overhead, and enable increased speed of analysis, decreased sample and solvent consumption and the possibility of increased detection efficiency.

Accordingly, several approaches have been developed in connection with miniaturization of devices for use in chemical analysis, particularly in micro-column liquid chromatography (μLC), wherein columns with diameters of 100 to 200 microns are used, in capillary electrophoresis (CE), wherein electrophoretic separation is conducted in capillaries on the order of 25 to 100 microns in diameter, and in microchannel electrophoresis (MCE), wherein electrophoresis is carried out within a microchannel on a substantially planar substrate. The conventional approach in miniaturization technology as applied to CE and μLC involves use of a silicon-containing material, i.e., a capillary fabricated from fused silica, quartz or glass. With MCE, an attractive method that is useful in conjunction with high throughput applications and enables reduction in overall system size relative to CE, miniaturized devices have been fabricated by silicon micromachining or lithographic techniques, e.g., microlithography, molding and etching. See, for example, U.S. Pat. Nos. 6,194,900 to Freeman et al., 6,093,362 to Kaltenbach et al., 6,033,628 to Kaltenbach et al., 5,804,022 to Kaltenbach, and 5,571,410 to Swedberg et al.; Fan et al. (1994) Anal. Chem. 66(1):177-184; Manz et al. (1993) Adv. in Chrom 33:1-66; Harrison et al. (1993) Sens. Actuators, B B 10(2):107-116; Manz et at. (1991) Trends Anal. Chem. 10(5):144-149; and Manz et at. (1990) Sensors and Actuators B (Chemical) B1(1-6):249-255. The use of micromachining techniques to fabricate miniaturized separation systems in silicon provides the practical benefit of enabling mass production of such systems, and there are a number of techniques that have now been developed by the microelectronics industry for fabricating microstructures from silicon substrates. Examples of such micromachining techniques to produce miniaturized separation devices on silicon or borosilicate glass chips can be found in U.S. Pat. Nos. 5,194,133 to Clark et al., 5,132,012 to Miura et al., 4,908,112 to Pace, and 4,891,120 to Sethi et al.

### SUMMARY OF THE INVENTION

Accordingly, it is a primary object of the invention to provide sol-gel matrices with entrapped biomolecules in a microfluidic format suitable for high throughput screening assays.

It is a further object of the invention to provide sol-gel biomatrices that are formed and then crushed to particles of appropriate size for forming beds in a microfluidic format suitable for high throughput screening.

It is yet another object of the invention to provide sol-gel biomatrices that are formed in situ within a microfluidic format.

It is a further object of the invention to provide sol-gel matrices formed as an integral part of a microfluidic device, for example, a microchip.

It is also an object of the invention to provide devices formed with a biomolecule-entrapped sol-gel over three-dimensional nanostructures within or as part of an assay chamber.

It is another object of the invention to provide a sol-gel entrapped enzyme that is incorporated into a microfluidic format.

It is another object of the invention to provide a sol-gel entrapped receptor that is incorporated into a microfluidic format.

It is another object of the invention to provide a sol-gel entrapped antibody that is incorporated into a microfluidic format.

It is another object of the invention to provide a sol-gel entrapped genome fragment that is incorporated into a microfluidic format.

It is another object of the invention to provide a sol-gel entrapped DNA or RNA oligomer that is incorporated into a microfluidic format.

It is yet another object of the invention to provide a microfluidic device using a sol-gel entrapped biomolecule, wherein one or more low molecular weight ligands are allowed to simultaneously interact with the biomolecule in the sol-gel while flowing across the sol-gel, and wherein the ligands that elute from the microfluidic device are analyzed using mass spectrometry, and wherein the ability of the ligands to interact with the entrapped biomolecule is determined by the change in the concentration of ligand relative to the concentration of ligand entering the microfluidic device.

It is another object of the invention to provide a microfluidic device incorporating a sol-gel entrapped biomolecule, wherein pores of varying size are present in the matrix so that the molecular weight of ligands that may contact the biomolecule can be controlled, wherein said ligands are caused to flow onto and/or into the microfluidic device.

It is an object of the invention to provide control over pore sizes and porosity of the biomolecule-entrapped sol-gel matrix using conditions of pH and temperature during the curing process so that the biomolecule is entrapped within pores of defined size and porosity and therefore defined substrate size limitation.

Additional objects, advantages and novel features of the invention will be set forth in part in the description which follows, and in part will become apparent to those skilled in the art upon examination of the following, or may be learned by practice of the invention.

The devices and methods of the invention claimed herein solve the above-mentioned problems and provide a method of immobilizing biomolecules by entrapment within an inorganic silicate or sol-gel for purposes of high throughput screening. The entrapped biomolecules are not altered by covalent modification, and are not merely adsorbed, and therefore will not leach from the matrix. The biomolecules entrapped within the sol-gel are used to perform binding or enzyme activity assays, for example, on a “chip” rather than keeping them as an off-line process that feeds into a micro device. The immobile molecule may be placed into a chamber on-device or may be formed in situ as a part of the device.

The present invention further provides devices and methods for performing high throughput screening using the biomolecule containing matrices.

The present invention further provides devices and methods using biomolecule containing silicate matrices as a packing material for microchannels and microcolumns in microfluidic devices to be used in a high throughput screening application.

The present invention further provides devices and methods using biomolecule containing silicate matrices as an integral part of the structure of microfluidic devices to be used in a high throughput screening application.

The present invention further provides devices and methods of using biomolecule-containing silicate matrices as a porous coating over three-dimensional structures of microfluidic devices to be used in a high throughput screening application.

The present invention further provides devices and methods for preparing the sol-gel immobilized biomolecules with specifically recited reaction parameters, e.g., conducting the reaction at a sufficiently low temperature to avoid too fast a reaction, and/or adding the biomolecule in base to the acidic sol-gel reaction mixture, which also facilitates control over reaction rate and promotes reaction homogeneity.

The present invention further provides devices and methods for carrying out an enzymatic digestion method using a silicate-immobilized enzyme in a format that ensures correspondence between the pore size of the matrix and the molecular size of the reagents used and/or the potential substrates to be tested. In particular, the invention provides devices and methods for a high throughput method using small reactants of molecular size having molecular weights of less than about 3000 Da. The claimed invention provides a match between the pore size and the reactant size, and the silicate-entrapped biomolecules are an ideal media for accomplishing in situ digests and is adaptable to high throughput procedures. Therefore, the devices and methods described herein are ideal for the high throughput procedures used in the pharmaceutical, biotechnology, agricultural or chemical industries, for example, or any other endeavor wherein entrapped biomolecules could be used on-line or on-device for assays or drug discovery.

The entrapped biomolecules are those such as receptors, antibodies, enzymes, gene and genome fragments, DNA and RNA oligomers, extracellular or secreted proteins, or polysaccharides or glycoproteins or lipids, plant products, fermentation products, membrane fragments or solubilized membrane proteins, and the like, without limitation. Such silicate-entrapped biomolecules are particularly suited for use with microfluidic devices, and offer considerable advantages over presently available technologies for immobilizing biomolecules for use in such devices and processes.

### BRIEF DESCRIPTION OF THE DRAWINGS

FIG. 1 shows a schematic diagram of protein entrapment in a sol-gel during polymerization.

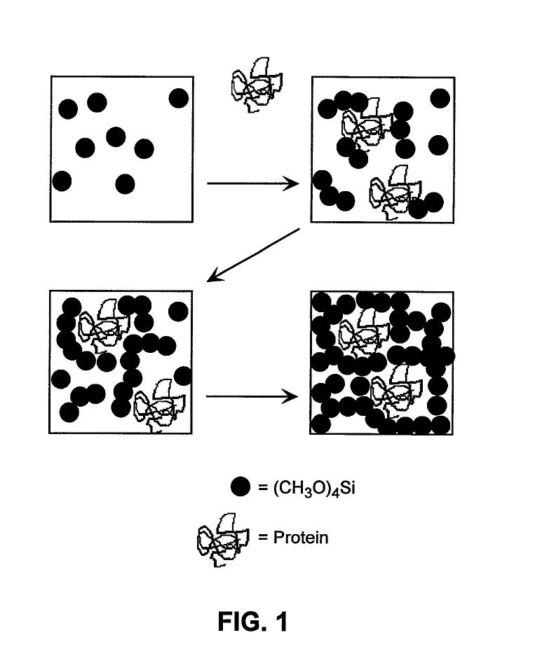

FIG. 2 shows a comparison of CE electropherograms of cytochrome c (˜13,000 Da) digestion products , wherein the digestions were performed using either trypsin in solution or trypsin sol-gel. Both digestions occurred for 18 hours at 370° C.

FIG. 3 shows a comparison between the CE electropherograms of Bombesin, either intact or digested for 1 minute with 1 mg trypsin sol-gel.

FIG. 4 shows CE electropherograms of bombesin fragments resulting from 3 minute digests using 10 mg trypsin sol-gel. The same portion of trypsin sol-gel was used for 11 repetitions and shows that activity is retained for extended uses of the sol-gel.

FIG. 5 shows a comparison of CE electropherograms of bombesin fragments resulting from 3 minute digests with 10 mg trypsin sol-gel, when the trypsin sol-gel was re-used 11 times versus when it was re-used 20 times.

### DETAILED DESCRIPTION OF THE INVENTION

### I. Definitions and Nomenclature

Before the present invention is described in detail, it is to be understood that unless otherwise indicated this invention is not limited to specific antibodies, receptors, or enzymes, or the like, as such may vary. It is also to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise. Thus, for example, reference to “an antibody” includes two or more antibodies, reference to “a complex” includes two or more complexes, and so forth.

The term “matrix” or “matrices” refers to the inorganic host that forms the body of the composite containing the biomolecules. The term “bed” refers to a preparation using the sol-gel matrix crushed and formed into particles of defined size, useful for proteolytic digestion or other enzyme or receptor activity, for example, and providing additional surface area for reaction. The term “nanostructure” refers to a structure formed from the matrix and biological material entrapped therein, predominantly of a size less than 100 nm. The term “xerogel” refers to a dried gel, which contains no liquid and is highly porous.

The term “fluid” as used herein refers to matter that is nonsolid or at least partially gaseous and/or liquid. A fluid may contain a solid that is minimally, partially or fully solvated, dispersed or suspended. Examples of fluids include, without limitation, aqueous liquids (including water per se and salt water) and nonaqueous liquids such as organic solvents and the like.

The term “gel” refers to a colloidal solution, more specifically a liquid in a solid. It begins in liquid form, and becomes free-standing as a result of a rapid increase of viscosity and polymerization. As it becomes viscous, it can be formed, cast, molded, shaped, spun or drawn into desired shapes. Preferred shapes for the present invention include, but are not limited to, films, fibers, monoliths, pellets, granules, tablets, rods or bulk. Particularly preferred shapes include thin-films and coatings, because gelation is enhanced, i.e., the thin layer encourages the rapid formation of the gel state (the “aged, aqueous gel” or “wet gel”) from the liquid state (the “viscous aqueous mixture”). In certain embodiments, it is desirable that the gel form and then be crushed into small particulates, suitable for forming beds to be incorporated into a microfluidic device. Sizes of particulates suitable for forming beds are from about 10 to about 80 microns in diameter. Alternatively, the gel may be formed in small particulates of desired shape and dimension directly by spraying droplets, forming a mist, or by use of a mold of desired shape and dimension in which the gel hardens.

However, if the material is too fluid, it cannot retain the selected shape; but if it is too viscous, it may be difficult to form into thin films or fibers. After the “aged” gel assumes its free-standing properties, it is dried over a period of time under select conditions to lock the conformation of the gel, its pores, matrices and interconnecting channels into fixed positions and permit long term storage (the “dried gel”).

The term “monolith” means a solid-like body in visibly one piece, and may be from several um in size to greater than tens of mm in size and beyond. The term “thin film” means a supported or unsupported layer of a thickness from 30 nm to 0.1 mm with a substantially larger cross section, typically from several mm in size and beyond.

The term “microanalytical device” refers to a device having features of micron or submicron dimensions, and that can be used in any number of chemical processes involving very small amounts of fluid. The term microanalytical device is used interchangeably with the term microfluidic device. “Optional” or “optionally” means that the subsequently de-scribed circumstance may or may not occur, so that the description includes instances where the circumstance occurs and instances where it does not. For example, recitation of a step as “optional” encompasses both a method that includes the step as well as a method that does not include the step.

### II. Sol-Gel Technology

The devices and methods of the invention claimed herein provide an improved method of immobilizing biomolecules, wherein the biomolecules are immobilized by entrapment within an inorganic silicate or sol-gel.

The sol-gel process is an important route for advanced metal-oxide glasses and ceramics. The method is currently used or of potential use for protective, optical and electronic coatings, optical fiber preforms, nonlinear optical devices, dielectrics or superconductors, display materials, and structures. The sol-gel technique provides a relatively low temperature, controlled method of producing a large variety of shapes such as monodispersed particles, uniform coatings, fibers, dense or porous articles, and mixed metal oxides having controlled stoichiometry and purity at the molecular level.

The sol-gel process has been based mostly on the same group of starting materials, the metal alkoxides, carboxylates and diketonates. These precursors are hydrolyzed, then condensed in the presence of an alcohol/water solution to form a gel, which is then dried and fired to give the final product. Chemical control of product formation is manipulated by temperature, type of catalyst and pH as well as by the type and ratio of reactants in solution. See, e.g., C. J. Brinker et al., in “Ultrastructure Processing of Ceramics, Glasses and Composites I” (1984), at pp. 43 et seq.

Thus, the reaction procedure controls to a large extent the morphology of the final gel, and, therefore, the final ceramic microstructure as well. Low water content and/or acidic conditions will give spinnable gels because the precursor polymer will, as noted above, be substantially linear. Higher water content will give slightly crosslinked, coatable gels, while a very high water content and/or basic conditions will give highly crosslinked gel products that are useful in casting processes and for powder formation. See B. J. J. Zelinski et al. (1984), J Phys. Chem. Solids 45:1069, and L. C. Klein et al. (1985), Ann. Rev. Mat. Sci. 15:227. Alternatively a method of using an aqueous, alcohol-free sol-gel may be used which does not result in the release of alcohol in the course of the reaction. Thus, contact with alcohol can be eliminated throughout the reaction, thereby avoiding the alcohol-caused denaturation of many biopolymers (caused by chain unfolding or molecule aggregation), typically seen in the conventional encapsulation methods. Consequently, the types of biopolymers that can be incorporated in these composites are essentially unlimited.

### III Sol-Gel Immobilization of Biomolecules

The biomolecule is immobilized in the polymeric matrix using a sol-gel polymerization process. The process involves polymerization of suitable monomers at or near room temperature to form an inorganic polymeric matrix (e.g., glass), wherein suitable monomers include alkoxides and esters of metallic and semiconducting elements, with Si, Al, Ti, Zr, and P being preferred such elements. The most preferred monomers include silicon and have a silicon to oxygen ratio from about 1:2 to about 1:4.

For example, biomolecules can be immobilized in silica polymeric matrices by a sol-gel process such as the hydrolysis of tetramethoxysilane or another polyalkoxysilane that contains one or more silicon atoms. Condensation of the resulting silanol in the presence of the biomolecule results in entrapment of the biomolecule. Binding of biomolecules in silica or other inorganic polymeric matrices formed from sol-gels can stabilize the enzyme.

Sol-gel derived processing can be performed at low temperatures, i.e. approximately 40° C. or below, and at either low or high pH's. For the purpose of this application, low pH processing has been used. The conditions of low temperature and low pH can be important for uniformly incorporating the biomolecule and for maintaining the functionality of biomolecules incorporated into the sol-gel matrix. The advantages of sol-gel derived processing include the following: 1) a sol, which is a suspension of colloidal size particles, is in liquid form before it gels; 2) the entire reaction can be carried out at ambient temperature; and 3) the microporosity of sol-gel glasses can be controlled by, for example, varying water content, timing of proton addition, proton concentration, aging time, and drying time. Pore sizes may also be affected via the size of the colloidal particles chosen in the sol. Preparation of sol-gels under higher pH will promote particle growth and postpone the gelation steps. These preparations will generate monoliths with denser and larger building blocks that produce larger average pore diameters and specific surface area. When processing is carried out using very low pH conditions (<2), the polycondensation of silica particles generally yields a more narrow pore monolith. The pore sizes achievable with sol-gel processing in general are in the nanometer range. During the liquid phase of the reaction, the biomolecules can be added to the liquid sol before it gels. The molecules then become entrapped within the solid matrix.

The sol-gel process is acid-catalyzed; however, the biomolecules are added in a buffer solution having a higher pH. Increasing the pH on addition of the biomolecules helps to provide reaction control by preventing immobilization from occurring too quickly and improving uniformity of the reaction. Conducting the reaction at a lower temperature also provides these benefits. The final pH of the reaction mixture can be controlled as well, so that the pH remains in the realm needed for stability of any particular biomolecule. The pH should be one in which the biomolecule maintains its optimally active conformation while it becomes entrapped in the sol-gel.

The pores can be developed and controlled by packing (upon gelation) of colloidal particles of different size, and the pore size can be strongly related to the starting size of the colloidal particles (Iler in The Chemistry of Silica, Wiley, New York, 1979; and Shafer et al. (1987) J. Appl. Phys. 61(12):5438-5446. The colloidal particles generally range from about 1 nm to about 30 mn in diameter. When the polymerization process occurs under acidic conditions, the particle range is closer to about 1 to about 5 nm.

It is possible to achieve a desired pore size using the aqueous colloidal process under a constant value of solution pH. However, the alkoxide-based sol-gel process may also be used. In the present invention, pore size can be varied from 1 to 100 nm or more to accommodate biomolecules or complexes of biomolecules, or fragments of cells, of different sizes. The preferred size ranges from 2 to 50 nm. However, the pore size is generally dependent on the size of biomaterial to be entrapped. For example, if proteins are to be entrapped, the more preferred pore size will range from a few to several tens of nanometers. For DNA, it is of the order of 15 to 30 nm. For cells or cell fragments, the pore size may be even larger, ranging from 100 nm and above. Also, since permeability is dependent on pore size, it can also be varied over a broad range in the colloidal sol-gel process to affect the reaction kinetics of the resultant composites.

The invention further provides the method, wherein the size of the sol particle is selected to produce a pore size when the gel is dried which is essentially the same as the size of a molecule of the entrapped biological material.

In a particular embodiment, a method of making a porous, inorganic matrix containing a biological material entrapped therein comprises: (a) forming an aqueous composition comprising a ceramic oxide colloidal sol mixed with an acidified oxide salt solution, (b) adding to said composition an amount of the biological material in a physiologically acceptable-buffered solution wherein the resulting aqueous composition has a pH ranging from about 6 to about 8.5, said aqueous composition becoming turbid on being transformed into a polymerizing hydroxide solution and transforming to a gel; (c) shaping the gel produced in step (b) into a final form and aging the gel at a temperature of from about 4° C. to about 25° C. for about two weeks; (d) rinsing said gel during the aging period; and (e) optionally crushing said gel into a powder and storing under a suitable pH buffer until use; wherein molecules of said biological material are entrapped within pores of the gel. A suitable pH is one in which the biomolecule is stable, but not necessarily active. For example, for trypsin, a suitable pH might be 2.5.

In a preferred embodiment, the sol is comprised of colloidal silica sol and a dissolved metal silicate, e.g. sodium silicate. In certain other preferred embodiments, the sol is comprised a combination of a tetraalkyl orthosilicate such as tetra-ethyl orthosilicate (TEOS) or tetra-methyl orthosilicate (TMOS) with a silane substituted with at least two leaving groups (usually OR and halo) and a C₈-C₂₄ alkyl group (e.g., C18-silane), resulting in a hydrophobic gel. Such a hydrophobic gel is particularly well suited as a component of a microfluidic device, and can be formed and crushed into particles, forming a bed that can be incorporated into a microcolumn or microchannel. Alternatively, the hydrophobic gel can be caused to form into any shape desired as a component of the microfluidic device, such as a porous coating over some portion of the microfluidic device over which the analytes flow, or incorporated into a three dimensional nanostructure of the device.

The invention provides embodiments, wherein the aqueous gel is shaped, preferably into a monolithic gel, thin film, or fiber. The invention also provides embodiments, wherein the dried gel comprises pores having an average diameter ranging from 1 nm to 100 nm, or more preferably ranging from 2 nm to 50 nm. More specifically, the pores of the dried gel comprise a matrix having pores of approximately the same dimension as the molecules of biological material entrapped therein. Alternatively, in certain other embodiments, the pore sizes will be substantially smaller that the size of the entrapped biomolecule.

The invention further provides a porous, inorganic, colloidal sol-gel having entrapped therein an active biological material, wherein the sol-gel is prepared in accordance with the methods provided herein. In a preferred embodiment of the present invention, the sol-gel is prepared comprising colloidal silica sol and dissolved sodium silicate. The preferred entrapped biological material within the sol-gel is an RNA, DNA, or protein, or an active fragment of DNA, RNA, or proteins, as well as cells or tissues. The more preferred entrapped biological material is any active protein or active fragment thereof.

In yet further embodiments, the invention provides a method, wherein the change in one or more optical characteristics of the entrapped biological material is qualitatively or quantitatively measured by spectroscopy, utilizing one or more techniques selected from the group consisting of UV, IR, visible light, fluorescence, luminescence, absorption, emission, excitation and reflection.

In addition, the invention provides a method of storing a biological molecule or complex of molecules, or a protein or protein fragment or complex of proteins or protein fragments, in a porous, inorganic matrix, wherein the biological activity of the material is retained for long periods of time. The preferred porous inorganic matrix is a sol-gel, comprising colloidal silica sol and dissolved sodium silicate. In certain embodiments, the activity of the biomolecule is retained even under adverse conditions, such as could be encountered through the use of solvents and high pressures, for example. The activity of entrapped biomolecules may be enhanced in the sol-gel, relative to unentrapped biomolecules, as reported in Altstein et al. (2001), cited supra. The structural stability provided by the sol-gel matrix may provide stability to the entrapped biomolecule and resistance of the entrapped biomolecules (IgGs) to denaturation by solvents.

In certain other embodiments, where the activity of the biological material is sensitive to the preparation and storage conditions or to the conditions employed during use of the sol-gel, at least 10% of the biological activity is retained. In certain embodiments, the biological activity may be affected, such as a change in substrate specificity or a shift in K_{D}, for example, where the biological molecule is an enzyme. The activity of the entrapped biological molecule may vary from the reference activity as a result of the preparation and storage conditions in the sol-gel. Any variation in activity is not to be construed as affecting the operability of the entrapped sol-gel. For certain robust biological molecules, the activity is retained even under adverse conditions.

### IV. Biomolecules

The biomolecules useful in conjunction with the invention may be any organic molecule, whether naturally occurring, recombinantly produced, or chemically synthesized in whole or in part, wherein the biomolecule is, was, or can be a part of a living organism. Biomolecules encompass, for example, nucleotides, amino acids and monosaccharides, as well as oligomeric and polymeric species such as oligonucleotides and polynucleotides, peptidic molecules such as oligopeptides, polypeptides and proteins, saccharides such as disaccharides, oligosaccharides, polysaccharides, mucopolysaccharides or peptidoglycans (peptido-polysaccharides) and the like. Biomolecules also include antibodies, ribosomes, enzymes, enzyme cofactors, extracellular or secreted proteins, viral or bacterial gene products, plant products, fermentation products, membrane fragments or solubilized membrane proteins, gene and genome fragments. The term “biomolecule” is also intended to encompass aggregates of biological molecules, such as biomolecule complexes or portions of cells or tissues. Examples of portions of cells comprise, for example, cellular fragments, intracellular organelles and solubilized membrane proteins, wherein the intracellular organelles or solubilized membrane proteins may include any cofactors or other associated cellular or membrane components necessary for full or native function. In some cases, it may be desirable to utilize such preparations lacking the associated cofactors or components. Examples of portions of tissues comprise, for example, excreted or secreted proteins or mucopolysaccharides, extracellular matrix proteins and mucins, but are not limited to these examples.

It will be appreciated that, as used herein, the terms “nucleoside” and “nucleotide” refer to nucleosides and nucleotides containing not only the conventional purine and pyrimidine bases, i.e., adenine (A), thymine (T), cytosine (C), guanine (G) and uracil (U), but also protected forms thereof, e.g., wherein the base is protected with a protecting group such as acetyl, difluoroacetyl, trifluoroacetyl, isobutyryl or benzoyl, and purine and pyrimidine analogs. Suitable analogs will be known to those skilled in the art and are described in the pertinent texts and literature. Common analogs include, but are not limited to, 1-methyladenine, 2-methyladenine, N⁶-methyladenine, N⁶-isopentyladenine, 2-methylthio-N⁶-isopentyladenine, N,N-dimethyladenine, 8-bromoadenine, 2-thiocytosine, 3-methylcytosine, 5-methylcytosine, 5-ethylcytosine, 4-acetylcytosine, 1-methylguanine, 2-methylguanine, 7-methylguanine, 2,2-dimethylguanine, 8-bromoguanine, 8-chloroguanine, 8-aminoguanine, 8-methylguanine, 8-thioguanine, 5-fluorouracil, 5-bromouracil, 5-chlorouracil, 5-iodouracil, 5-ethyluracil, 5-propyluracil, 5-methoxyuracil, 5-hydroxymethyluracil, 5-(carboxyhydroxymethyl)uracil, 5-(methylaminomethyl)uracil, 5-(carboxymethylaminomethyl)-uracil, 2-thiouracil, 5-methyl-2-thiouracil, 5-(2-bromovinyl)uracil, uracil-5-oxyacetic acid, uracil-5-oxyacetic acid methyl ester, pseudouracil, 1-methylpseudouracil, queosine, inosine, 1-methylinosine, hypoxanthine, xanthine, 2-aminopurine, 6-hydroxyaminopurine, 6-thiopurine and 2,6-diaminopurine. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. As used herein, the term “oligonucleotide” shall be generic to polydeoxynucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones (for example, PNAs), providing that the polymers contain nucleobases in a configuration that allows for base pairing and base stacking, such as is found in DNA and RNA. Thus, these terms include known types of oligonucleotide modifications, for example, substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.). There is no intended distinction in length between the terms “polynucleotide” and “oligonucleotide,” and these terms will be used interchangeably. These terms refer only to the primary structure of the molecule. As used herein the symbols for nucleotides and polynucleotides are according to the IUPAC-IUB Commission of Biochemical Nomenclature recommendations (Biochemistry 9:4022, 1970).

The terms “peptide,” “peptidyl” and “peptidic” as used throughout the specification and claims are intended to include any structure comprised of two or more amino acids. For the most part, the peptides that may be entrapped in the present devices comprise about 5 to 10,000 amino acids, preferably about 5 to 1000 amino acids. The amino acids forming all or a part of a peptide may be any of the twenty conventional, naturally occurring amino acids, i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y). Any of the amino acids in the peptidic molecules forming the present devices may be replaced by a non-conventional amino acid. In general, conservative replacements are preferred. Conservative replacements substitute the original amino acid with a non-conventional amino acid that resembles the original in one or more of its characteristic properties (e.g., charge, hydrophobicity, steric bulk; for example, one may replace Val with Nval). The term “non-conventional amino acid” refers to amino acids other than conventional amino acids, and include, for example, isomers and modifications of the conventional amino acids (e.g., D-amino acids), non-protein amino acids, post-translationally modified amino acids, enzymatically modified amino acids, constructs or structures designed to mimic amino acids (e.g., a,a-disubstituted amino acids, N-alkyl amino acids, lactic acid, y-alanine, naphthylalanine, 3-pyridylalanine, 4-hydroxyproline, O-phosphoserine, N-acetylserine, N-formylmethionine, 3-methylhistidine, 5-hydroxylysine, and norleucine), and peptides having the naturally occurring amide —CONH—linkage replaced at one or more sites within the peptide backbone with a non-conventional linkage such as N-substituted amide, ester, thioamide, retropeptide (—NHCO—), retrothioamide (—NHCS—), sulfonamido (—SO₂NH—), and/or peptoid (N-substituted glycine) linkages. Accordingly, the peptidic molecules of the array include pseudopeptides and peptidomimetics. The peptides of this invention can be (a) naturally occurring, (b) produced by chemical synthesis, (c) produced by recombinant DNA technology, (d) produced by biochemical or enzymatic fragmentation of larger molecules, (e) produced by methods resulting from a combination of methods (a) through (d) listed above, or (f) produced by any other means for producing peptides.

Examples of various peptidyl compounds include, but are not limited to, the following:

Coagulation modulators include α₁-antitrypsin, α₂-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, thrombomodulin and combinations thereof.

Cytokines include, but are not limited to, the following: transforming growth factors (TGFS) such as TGF-β1, TGF-β2, and TGF-β3; bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), heparin-binding neurotrophic factor (HBNF), and insulin-like growth factor (IGF)); connective tissue activated peptides (CTAPs), osteogenic factors; colony stimulating factor; interferons, including interferon-α, interferon α-2a, interferon α-2b, interferon α-n3, interferon-β, and interferon-γ; interleukins, including interleukin-1, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-15, interleukin-16, and interleukin-17; tumor necrosis factor; tumor necrosis factor-α; granuloycte colony-stimulating factor (G-CSF); granulocyte-macrophage colony-stimulating factor (GM-CSF); macrophage colony-stimulating factor; Inhibins (e.g., Inhibin A and Inhibin B); growth differentiating factors (e.g., GDF-1); Activins (e.g., Activin A, Activin B, and Activin AB); midkine (MD); and thymopoietin.

Endorphins are peptides that activate opiate receptors. Agonist and antagonist derivatives of the naturally occurring endorphins are also contemplated. Representative examples of endorphins and pharmacologically active endorphin derivatives include dermorphin, dynorphin, α-endorphin, β-endorphin, γ-endorphin, σ-endorphin [Leu⁵]enkephalin, [Met⁵]enkephalin, substance P, and combinations thereof.

Peptidyl hormones include, but are not limited to, activin, amylin, angiotensin, atrial natriuretic peptide (ANP), calcitonin (derived from chicken, eel, human, pig, rat, salmon, etc.), calcitonin gene-related peptide, calcitonin N-terminal flanking peptide, cholecystokinin (CCK), ciliary neurotrophic factor (CNTF), corticotropin (adrenocorticotropin hormone, ACTH), corticotropin-releasing factor (CRF or CRH), follicle-stimulating hormone (FSH), gastrin, gastrin inhibitory peptide (GIP), gastrin-releasing peptide, glucagon, gonadotropin-releasing factor (GnRF or GNRH), growth hormone releasing factor (GRF, GRH), human chorionic gonadotropin (hCH), inhibin A, inhibin B, insulin (derived from beef, human, pig, etc.), leptin, lipotropin (LPH), luteinizing hormone (LH), luteinizing hormone-releasing hormone (LHRH), lypressin, α-melanocyte-stimulating hormone, β-melanocyte-stimulating hormone, γ-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatostatin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, triiodothyronine, vasoactive intestinal peptide (VIP), and vasopressin (antidiuretic hormone, ADH).

Particularly preferred analogues of LHRH include buserelin, deslorelin, fertirelin, goserelin, histrelin, leuprolide (leuprorelin), lutrelin, nafarelin, tryptorelin and combinations thereof.

Kinins include bradykinin, potentiator B, bradykinin potentiator C, and kallidin and combinations thereof.

Enzymes include transferase, RNase, DNase, telomerase, ligase, nuclease, ribonuclease; hydrogenase, dehydrogenase, aldase, amidase, aminotransferase, amylase, anhydrase, apyrase, arginase, aspartase, aspariginase, carboxylase, carboxypeptidase, catalase, cellulase, cholinesterase, acetylcholinesterase, deaminase, dextranase, dismutase, elastase, esterase, fumarase, glucosidase, hexokinase, isomerase, invertase, kinase, lactasee, lipase, lysozyme, malase, naringinase, oxidase, oxygenase, papain, pectinase, peptidase, pepsin, peroxidase, phosphodiesterase, phosphotase, protease, reductase, transferase, tyrosinase, urase, trypsin, chymotrypsin, hydrolases, isomerases, proteases, ligases and oxidoreductases such as esterases, phosphatases, glycosidases and peptidases. Specific examples of enzymes include superoxide dismutase (SOD), tissue plasminogen activator (TPA), renin, adenosine deaminase, alpha-glucocerebrosidase, asparaginase, domase-alpha, hyaluronidase, elastase, trypsin, thymidine kinase (TK), tryptophan hydroxylase, urokinase, kallikrein, bromelain, cathepsins B, D, G, C, clostripain, endoproteinase Arg C, endoproteinase Asp N, endoproteinase Glu C, endoproteinase Lys C, Factor Xa, proteinase K, subtilisin, thermolysin, acyloamino acid releasing enzyme, aminopeptidases, carboxypeptidases, pyroglutamate aminopeptidase, and combinations thereof.

Enzyme inhibitors include leupeptin, chymostatin, pepstatin, renin inhibitors, angiotensin converting enzyme (ACE) inhibitors, and the like.

Antibodies include both monoclonal and polyclonal antibodies, as well as antibody fragments, such as the F(ab′)₂, Fab, Fv and Fc fragments of monoclonal antibodies.

Peptidyl drugs: Peptidyl drugs in addition to those mentioned above include abarelix, anakinra, ancestim, bivalirudin, bleomycin, bombesin, desmopressin acetate, des-Q14-ghrelin, enterostatin, erythropoeitin, exendin-4, filgrastim, gonadorelin, insulinotropin, lepirudin, magainin I, magainin II, nerve growth factor, pentigetide, thrombopoietin, thymosin α-1, and urotensin II and combinations thereof.

### V. Microfluidic Systems

The term “microanalytical device” refers to a device having features of micron or submicron dimensions, and that can be used in any number of chemical processes involving very small amounts of fluid. Such processes include, but are not limited to, electrophoresis (e.g., CE or MCE), chromatography (e.g., μLC), screening and diagnostics (using, e.g., hybridization or other binding means), and chemical and biochemical synthesis (e.g., DNA amplification as may be conducted using the polymerase chain reaction, or “PCR”). The features of the microanalytical devices are adapted to the particular use. For example, microanalytical devices that are used in separation processes, e.g., MCE, contain microchannels (termed “microcolumns” herein when enclosed) on the order of 1 μm to 200 μm in diameter, typically 10 μm to 75 μm in diameter, and approximately 0.1 to 50 cm in length. Microanalytical devices that are used in chemical and biochemical synthesis will generally contain reaction zones (termed “reaction chambers” herein when enclosed, i.e., again, when the cover plate is in place on the microchannel-containing substrate surface) having a volume of from about 1 nl to about 500 μl, typically from about 10 μl to about 200 μl, or may be scaled to use volumes of from about 10 nl to about 20 μl.

The term “microfluidic devices” refers to two and three dimensional microanalytical devices such as microarrays (e.g., “chips”), microchannels or microcolumns, as described in copending U.S. patent application Ser. No. 09/502,593 to Tso et al. and in U.S. Pat. Nos. 6,194,900 to Freeman et al., 6,093,362 to Kaltenbach et al., 6,033,628 to Kaltenbach et al., 5,804,022 to Kaltenbach, 5,571,410 to Swedberg et al., 6,176,962 to Soane et al., and 6,103,199 to Soane et al.

The microfluidic structure of the present devices may be designed to utilize electrophoresis, chromatography, electrochromatography, capillary chromatography, micro-reaction cavity procedures, miniaturized liquid communication, biosensor flow cells, but is not limited to these examples. Reaction cavities constructed in accordance with the invention may, for example, be used for binding assays or for various forms of solid phase synthesis, such as peptide or oligonucleotide synthesis, PCR, DNA-solid phase sequencing reactions, just to mention a few. The devices may also be designed to interface with various analytical and/or detection devices, such as a mass spectrometer, fluorescence detector, fluorescence polarization, radioactivity, UV or visible or infrared spectroscopy, or any other detection device that is used with non-microfluidic systems.

The term “liquid phase analysis” is used to refer to any analysis that is carried out on a solute in the liquid phase. Accordingly, “liquid phase analysis” as used herein includes chromatographic separations, electrophoretic separations, and electrochromatographic separations. The general term “analysis” refers to characterization of a sample or identification of one or more components therein, and is distinct from a chemical or biochemical “process” in which a material is chemically or biochemically altered to produce a desired product.

“Chromatography” generally refers to preferential separations of components, and includes reverse-phase, hydrophobic interaction, ion exchange, molecular sieve chromatography, and like methods.

“Electrophoresis” refers to the migration of particles or macromolecules having a net electric charge where said migration is influenced by an electric field. Accordingly, electrophoretic separations include separations performed in columns packed with gels as well as separations performed in solution.

“Electrochromatography” separations refer to separations effected using a combination of electrophoretic and chromatographic techniques. Exemplary electrochromatographic separations include packed column separations using electromotive force (Knox et al. (1987) Chromatographia 24:135; Knox et al. (1989) J. Liq. Chromatogr 12:2435; Knox et al. (1991) Chromatographia 32:317), and micellar electrophoretic separations (Terabe et al. (1985) Anal. Chem. 57:834-841).

For example, a simple microfluidic device comprises:

a substrate having first and second substantially planar opposing surfaces, with a cavity and at least one microchannel formed in the first planar surface, wherein the cavity serves as a reaction zone that is in fluid communication with each microchannel;

a cover plate arranged over the first planar surface, which in combination with the cavity defines a reaction chamber, and with each microchannel defining a microcolumn; and

at least one inlet port and at least one outlet port communicating directly or indirectly with the reaction chamber, enabling the passage of fluid from an external source into and through the reaction chamber,

wherein the substrate and the cover plate are comprised of a material that is thermally and chemically stable and resistant to biofouling.

The microfluidic devices also provide a design wherein a reaction fluid is introduced into the reaction chamber through the inlet port, either directly or indirectly (i.e., the inlet port may be in direct communication with the reaction chamber or with an upstream microchannel feeding into the chamber), the desired reaction is conducted in the reaction chamber, and the product of the reaction is collected upon removal from the device through the outlet port. Microchannels present in fluid communication with the reaction chamber may be used to increase the concentration of a particular analyte or chemical component prior to processing in the reaction chamber, to remove potentially interfering sample or reaction components, to conduct preparative processing prior to chemical processing in the reaction chamber, and to isolate and purify the desired product.

A detection means, i.e., any means, structure or configuration that allows one to interrogate a sample within a microanalytical device using analytical detection techniques, may also be incorporated into or operatively connected to the microfluidic device. Thus, a detection means can comprise one or more openings that communicate with, for example, a reaction chamber or microchannel, and allow an external detection device to be interfaced with the chamber or microchannel to detect an analyte therein. By the arrangement of two detection means opposite each other relative to the reaction chamber or the like, a “detection path” is formed, allowing detection of analytes passing through the reaction chamber using detection techniques well known in the art. An “optical detection path” refers to a configuration or arrangement of detection means to form a path whereby electromagnetic radiation is able to travel from an external source to a means for receiving radiation, wherein the radiation traverses the reaction chamber, microchannel, or the like. In this configuration, analytes passing through the microanalytical device can be detected via transmission of radiation orthogonal to the direction of fluid flow. A variety of external optical detection techniques can be readily interfaced with the present microanalytical devices, including, but not limited to, UV/Visible, Near IR, fluorescence, refractive index (RI) and Raman techniques.

Additional detection means and diagnostic means may be employed, either in addition to the optical techniques discussed above, or as an alternative means of detection and analysis. For example, detection using mass spectrometry or conductance or thermal conductivity may be employed. Some portion of the analytes may be diverted from the main flow path to the detection means, or the entire portion of the analytes may flow directly into the detection means.

The microdevices are customizable to any particular assay. No particular enzyme, antibody or receptor, for example, is required in order for the devices to work. Further, multiple lanes are possible, for performing assays and analyses in parallel, further saving time. In addition, the devices are reusable. Solvent conditions may be changed so that reactants are released from the equilibration bed if that is desired, for example, as described in Anal. Chem. (2001) Vol. 73, No. 11, page 2461. Therefore, the devices can be used to separate and detect ligands, for example, that fail to interact with a receptor or enzyme, and at a later time, the bound ligands can be dissociated from the receptor or enzyme, for example, and analyzed.

### VI. Methods of Use

The present invention further provides methods of using the above-described biomolecule-containing silicate matrices as a packing material for microchannels and microcolumns in microfluidic devices. The sol-gel may be formed into a particulate material, for example, by crushing the gel, resulting in the formation of particles such as beads that may then be incorporated into the microfluidic devices herein described. Preferably, the beads are from about 10 μm to about 80 μm in diameter.

The present invention further provides methods for performing high-throughput screening using the biomolecule containing matrices. For example, the invention may be used in conjunction with methods for carrying out enzymatic catalysis or receptor binding using silicate-immobilized enzymes and receptors. In particular, the present invention provides a format that ensures correspondence between the pore size of the matrix and the molecular size of the reagents used and/or the potential substrates to be tested. The entrapped biomolecules are not leached out due to the pore size of the sol-gel matrix. However substrates, reactants, ligands, and the like, may diffuse into the pores of the sol-gel matrix and gain access to the entrapped biomolecule and interact with the active sites or ligand binding sites, for example, of the biomolecule. In particular, the invention provides methods for a high throughput method using small reactants of molecular size having molecular weights of less than about 3000 Da. The claimed invention provides a match between the pore size and the reactant size. The silicate-entrapped biomolecules are an ideal medium for accomplishing in situ digests and a device incorporating the biomolecules is adaptable to high throughput procedures. Therefore, the devices and methods described herein are ideal for the high throughput procedures used in the pharmaceutical, biotechnology, agricultural or chemical industries, for example, or any other endeavor wherein entrapped biomolecules could be used on-line or on-device for assays or drug discovery.

The biomolecules may include any of those described in Section IV. In certain preferred embodiments, the protein or fragment thereof is an enzyme or active compound selected from, but not limited to, the group consisting of any RNase, DNase, telomerase, ligase, nuclease, ribonuclease, hydrogenase, dehydrogenase aldase amidase, aminotransferase, amylase, anhydrase, apyrase, arginase, aspartase, aspariginase, carboxylase, carboxypeptidase, catalase, cellulase, cholinesterase, acetylcholinesterase, deaminase, dextranase, dismutase, elastase, esterase, fumarase, glucosidase, hexokinase, isomerase, invertase, kinase, lactase, lipase, lysozyme, malase, naringinase, oxidase, oxygenase, papain, pectinase, peptidase, pepsin, peroxidase, phosphodiesterase, phosphatase, protease, reductase, transferase, tyrosinase, urease, trypsin, chymotrypsin, and combinations thereof.

In addition, the invention provides for the use of a sol-gel comprising an active biological material entrapped therein, to quantitatively or qualitatively detect a test substance that reacts with or whose reaction is catalyzed by said entrapped active biological material. The sol-gel thus may be used for binding assays or as a sensor, or may be useful in drug metabolism studies, without limitation.

Also provided is a method for the quantitative or qualitative detection of a test substance that reacts with or whose reaction is catalyzed by an active biological material, wherein said biological material is entrapped within a sol-gel, and wherein said sol-gel comprises a porous, inorganic matrix prepared by the following method: (a) preparing the sol-gel comprising said active biological molecule entrapped within a porous, inorganic matrix; (b) bringing said biological-molecule-containing sol-gel into contact with a gas or aqueous solution comprising the test substance; and (c) quantitatively or qualitatively detecting, observing or measuring the change in one or more optical characteristics in the biological material entrapped within the sol-gel.

Accordingly, the present invention relates to the conversion of assays into a microdevice format in which a biomolecule is entrapped inside an inorganic glass matrix and the matrix is incorporated into the device. In preferred embodiments, the biomolecule entrapped within the inorganic glass matrix is placed in a chamber within the device to create microchannels or microcolumns.

In one embodiment, low molecular weight reactants are equilibrated over these microchannels or microcolumns, and the reactants then proceed through a chromatographic purification and separation step. The output of the chromatographic step feeds into a mass spectrometer as the detector, so the detection involves relatively small masses. Optionally, deconvolution software can be incorporated into the system to resolve larger or more complex structures. However, one advantage of this embodiment of the invention is that low molecular weight reactants do not require deconvolution and the ligand binding with greatest affinity to the entrapped biomolecule is readily apparent, by its absence. Also since the low molecular weight reactants are being analyzed using mass spectrometry, very small differences in molecular weights of the reactants can be detected, and the entire assay is therefore made more sensitive. For example, ligands may be distinguished in the mass spectrometer having masses only 1 amu different. Finally, the entrapped biomolecule can release the bound low molecular weight reactant or ligand, and its identity can be determined directly, in order to verify the results inferred from analysis of the eluted ligands.

Sol-gels are particularly suited for use with microfluidic devices, and offer considerable advantages over presently available technologies for immobilizing biomolecules for use in such devices and processes. When the immobilized biomolecules are adapted for a micro format, the speed of the assays or reactions is substantially accelerated relative to conventional assays. Only a minimal amount of reagents is required and there is minimal diffusion time for equilibration over sol-gel microchannels or microcolumns. Therefore the kinetics of all aspects of the reaction is faster when miniaturized to the micro format scale.

The practice of the present invention will employ, unless otherwise indicated, conventional techniques of organic chemistry, polymer chemistry, silicate chemistry, biochemistry and the like, which are within the skill of the art. Such techniques are explained fully in the literature. All patents, patent applications, and publications mentioned herein, both supra and infra, are hereby incorporated by reference.

It is to be understood that while the invention has been described in conjunction with the preferred specific embodiments thereof, that the description above as well as the example that follows are intended to illustrate and not limit the scope of the invention. Other aspects, advantages and modifications within the scope of the invention will be apparent to those skilled in the art to which the invention pertains.

In the following example, efforts have been made to ensure accuracy with respect to numbers used (e.g., amounts, temperature, etc.) but some experimental error and deviation should be accounted for. Unless indicated otherwise, temperature is in degrees C and pressure is at or near atmospheric. All solvents were purchased as HPLC grade, and all reactions were routinely conducted under an inert atmosphere of argon unless otherwise indicated. All capillary electrophoreses were performed with a run buffer of 50 mM phosphate, pH 2.5. All chromatographic separations were performed using reverse phase HPLC over a C-18 stationary phase and with a mobile phase of increasing percentages of acetonitrile or methanol in water with 0.1% trifluoroacetic acid, unless noted otherwise. Unless otherwise indicated, the reagents used were obtained from the following sources: silanes, from Petrarch Systems, Inc., Bristol, Pa.; organic reagents including amines, from Aldrich Chemical Co., Milwaukee, Wis.; gases, from Matheson, Seacaucus, N.J.

### EXAMPLE

### (a) Preparation of Sol-gel With Entrapped Trypsin

Deionized water (1.69 g), 0.110 ml 0.04M HCl, and 7.39 ml (7.6 g) tetra-methyl orthosilicate (TMOS) were combined in a screw top vial and sonicated in an ice bath for 30 minutes. Aliquots of this solution (1.5 ml) were mixed with 1 ml trypsin (2 mM in ammonium bicarbonate buffer, pH 8.1) and placed into disposable cuvettes to age and cure. The solution became milky with the addition and became a gel within 30-40 seconds. Continued aging occurred at 4° C. for 2 weeks. The gel was rinsed twice daily with the buffer solution during the two week aging period. After the aging process, the gel was crushed to a powder and stored under a phosphate buffer (pH 2.5) in the refrigerator until use.

The entrapped trypsin sol-gel was tested for trypsin activity using various proteins as substrates. The following were completely digested using 10 mg of the sol-gel entrapped trypsin: bombesin (molecular weight 1619 Da), 1 minute at room temperature; insulin B chain (molecular weight 3500 Da) 10 minutes at room temperature; neurotensin (molecular weight 1700 Da) 1 hour at 37° C.; calcitonin (molecular weight 3000 Da) 18 hours at 37° C.; and cytochrome C (molecular weight 13,000 Da) 18 hours 37° C.

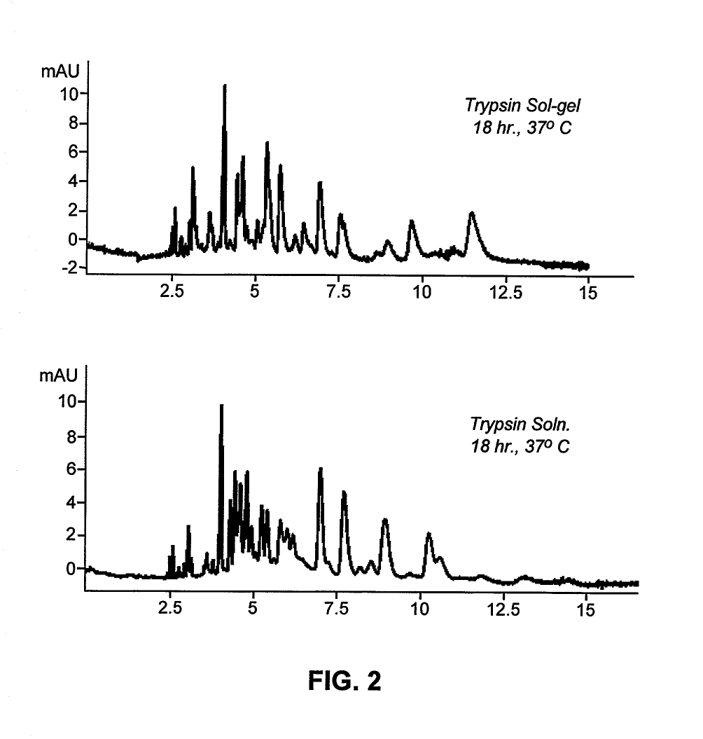

As shown in FIG. 2, the chromatographic patterns for cytochrome C digested with trypsin sol-gel and trypsin in solution at 37° C. for an identical amount of time were very similar, suggesting that the entrapped trypsin has retained its activity and substrate specificity.

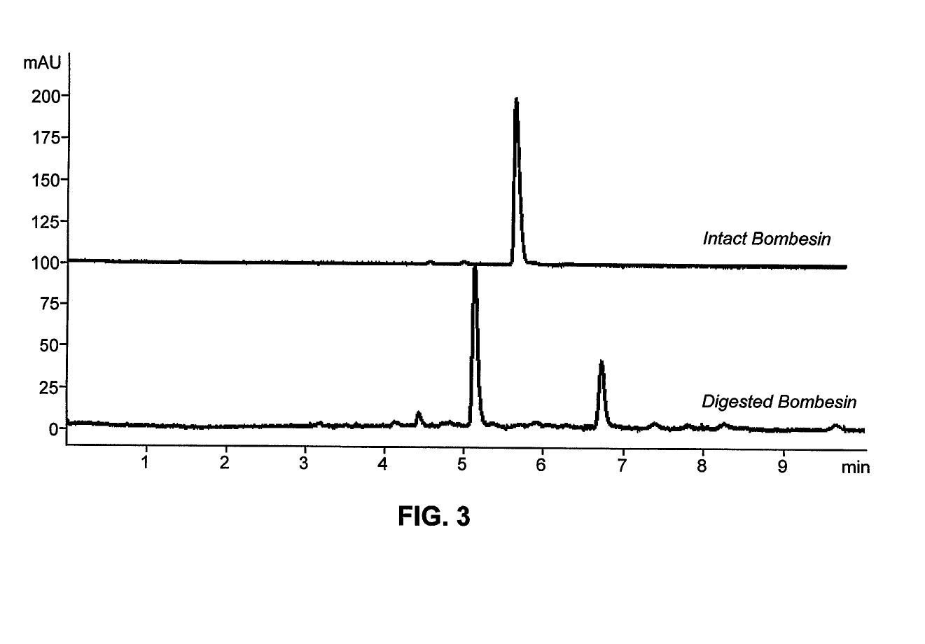

Similarly, FIG. 3 shows that even 1 minute of exposure to trypsin sol-gel was sufficient time to digest bombesin as shown by the CE electrophoregrams before and after exposure.

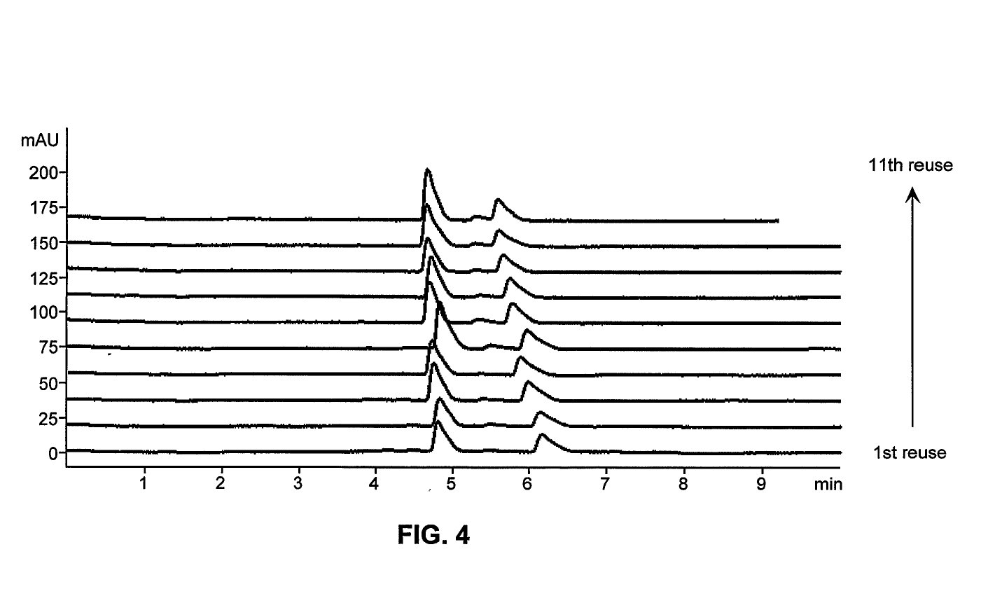

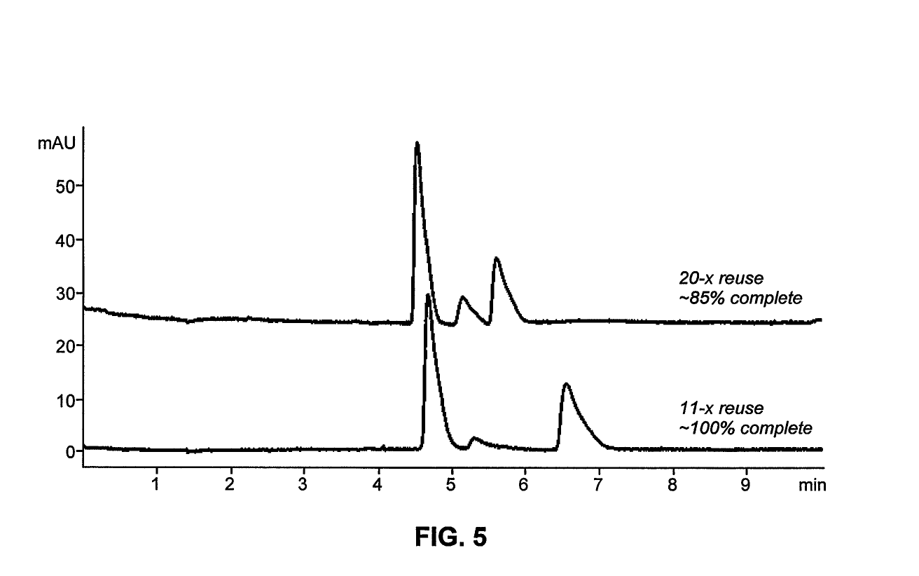

FIGS. 4 and 5 demonstrate that the trypsin sol-gel is stable and may be reused many times without apparent loss of enzyme activity: at least 85% activity is retained after reusing the trypsin sol-gel 20 times.

### (b) Microdevice Design

Designs for typical microassay device are shown and described in copending U.S. patent application Ser. No. 09/502,593 to Tso et al. and in U.S. Pat. Nos. 6,194,900 to Freeman et al., 6,093,362 to Kaltenbach et al., 6,033,628 to Kaltenbach et al., 5,804,022 to Kaltenbach, and 5,571,410 to Swedberg et al. Microchannels, microcolumns and/or microchambers of any of these devices are filled with an aqueous slurry of particulates of the sol-gel material containing trypsin. Sample analytes are allowed to flow through the sol-gel chamber and analyzed using a mass spectrometer, with sample introduction by electrospray.

We claim:

1. A method for immobilizing a biological molecule in a porous inorganic matrix, said method comprising:
forming an aqueous composition comprising a ceramic oxide colloidal sol mixed with an acidified oxide salt solution;
adding to said composition an amount of the biological molecule in a physiologically acceptable-buffered solution, said aqueous composition becoming turbid on being transformed into a polymerizing hydroxide solution and transforming to a gel;
shaping the gel produced in step (b) into a final form; and
aging the gel;
wherein said biological molecule is entrapped within pores of the gel, and the activity of the biological molecule is retained.

2. The method of claim 1, wherein the gel is aged for about two weeks.

3. The method of claim 1, wherein the gel is aged at a temperature of from about 4° C. to about 40° C.

4. The method of claim 1, wherein the pH of the mixture after the addition of the biological molecule is between about 6.0 and about 8.5.

5. The method of claim 1, wherein the pH of the mixture after the addition of the biological molecule is between about 4.0 and about 7.0.

6. The method of claim 1, wherein the pH of the mixture after the addition of the biological molecule is between about 7.0 and 9.0.

7. The method of claim 1, further comprising crushing the aged gel into particulates.

8. The method of claim 7, wherein the crushed gel particulates are between about 10 μm and about 80 μm in diameter.

9. A method for immobilizing a biological molecule in a porous inorganic matrix, said method comprising:
forming an aqueous composition comprising a ceramic oxide colloidal sol and a dissolved metal silicate mixed with an acidified oxide salt solution;
adding to said composition an amount of the biological material in a physiologically acceptable-buffered solution wherein the resulting aqueous composition has a pH ranging from about 6 to about 8.5, said aqueous composition becoming turbid on being transformed into a polymerizing hydroxide solution and transforming to a gel;
shaping the gel produced in step (b) into a final form; and
aging the gel;
wherein said biological molecule is entrapped within pores of the gel, and the activity of the biological molecule is retained.

10. The method of claim 9, wherein the sol is comprised of colloidal silica sol and a dissolved metal silicate.

11. The method of claim 9, wherein the metal silicate is sodium silicate.

12. The method of claim 9, wherein the sol comprises a tetraalkyl orthosilicate and a silane substituted with at least two leaving groups selected from the group consisting of OR and halo.

13. The method of claim 12, wherein the silane is substituted with a C₈-C₂₄ alkyl group.

14. The method of claim 13, wherein the alkyl group is C₁₈.

15. The method of claim 12, wherein the tetraalkyl orthosilicate is selected from the group consisting of tetra-ethyl orthosilicate (TEOS), tetra-methyl orthosilicate (TMOS), and combinations thereof.

16. The method of claim 1, wherein the sol is comprised of colloidal silica sol and a dissolved metal silicate.

17. The method of claim 16, wherein the metal silicate is sodium silicate.

18. The method of claim 1, wherein the sol comprises a tetraalkyl orthosilicate and a silane substituted with at least two leaving groups selected from the group consisting of OR and halo.

19. The method of claim 18, wherein the silane is substituted with a C₈-C₂₄ alkyl group.

20. The method of claim 19, wherein the alkyl group is C₁₈.

21. The method of claim 18, wherein the tetraalkyl orthosilicate is selected from the group consisting of tetra-ethyl orthosilicate (TEOS), tetra-methyl orthosilicate (TMOS), and combinations thereof.

22. The method of claim 12, further comprising crushing the gel into particulates.

23. The method of claim 22, wherein the crushed gel particulates are between about 10 μm to about 80 μm in diameter.

24. The method of claim 1, wherein the particle size of the ceramic oxide colloidal sol is selected to produce pores when the gel is aged, said pores being of a diameter which is approximately the same as the diameter of the biological molecule to be entrapped.

25. The method of claim 1, wherein the gel produced in step (b) is shaped into forms selected from the group consisting of a monolithic gel, thin film, or fiber.

26. The method of claim 24, wherein the pores have an average diameter ranging from about 1 nm to about 100 nm.

27. The method of claim 1, wherein the pores have an average diameter ranging from about 2 nm to about 50 nm.

28. The method of claim 9, wherein the particle size of the ceramic oxide colloidal sol is selected to produce pores when the gel is aged, said pores being of a diameter which is approximately the same as the diameter of the biological molecule to be entrapped.

29. The method of claim 28, wherein the diameter of the pores is less than the diameter of the entrapped biomolecule.

30. The method of claim 9, wherein the gel produced in step (b) is shaped into forms selected from the group consisting of a monolithic gel, thin film, or fiber.

31. The method of claim 9, wherein the pores have an average diameter ranging from about 1 nm to about 100 nm.

32. The method of claim 31, wherein the pores have an average diameter ranging from about 2 nm to about 50 nm.

33. The method of claim 24, wherein molecules having a mass of 3,000 Da or less can diffuse through the pores.

34. The method of claim 24, wherein molecules having a mass of 5,000 Da or less can diffuse through the pores.

35. The method of claim 24, wherein molecules having a mass of 10,000 Da or less can diffuse through the pores.

36. The method of claim 24, wherein molecules having a mass of 15,000 Da or less can diffuse through the pores.

37. The method of claim 28, wherein molecules having a mass of 3,000 Da or less can diffuse through the pores.

38. The method of claim 28, wherein molecules having a mass of 5,000 Da or less can diffuse through the pores.

39. The method of claim 28, wherein molecules having a mass of 10,000 Da or less can diffuse through the pores.

40. The method of claim 28, wherein molecules having a mass of 15,000 Da or less can diffuse through the pores.

41. The method of claim 1, wherein the biological molecule is selected from the group consisting of polynucleotides, enzymes, antibodies, coagulation modulators, cytokines, endorphins, peptidyl hormones, kinins, receptors, genes, gene fragments, cell fragments, membrane fragments, and solubilized membrane proteins.

42. The method of claim 41, wherein the enzyme is selected from the group consisting of RNase, DNase, telomerase, ligase, nuclease, ribonuclease; hydrogenase, dehydrogenase, aldase, amidase, aminotransferase, amylase, anhydrase, apyrase, arginase, aspartase, aspariginase, carboxylase, carboxypeptidase, catalase, cellulase, cholinesterase, acetylcholinesterase, deaminase, dextranase, dismutase, elastase, esterase, fumarase, glucosidase, hexokinase, isomerase, invertase, kinase, lactasee, lipase, lysozyme, malase, naringinase, oxidase, oxygenase, papain, pectinase, peptidase, pepsin, peroxidase, phosphodiesterase, phosphotase, protease, reductase, transferase, tyrosinase, urase, trypsin, chymotrypsin, hydrolases, isomerases, proteases, ligases and oxidoreductases such as esterases, phosphatases, glycosidases and peptidases, superoxide dismutase (SOD), tissue plasminogen activator (TPA), renin, adenosine deaminase, alpha-glucocerebrosidase, asparaginase, dornase-alpha, hyaluronidase, elastase, trypsin, thymidine kinase (TK), tryptophan hydroxylase, urokinase, kallikrein, bromelain, cathepsins B, D, G, C, clostripain, endoproteinase Arg C, endoproteinase Asp N, endoproteinase Glu C, endoproteinase Lys C, Factor Xa, proteinase K, subtilisin, thermolysin, acyloamino acid releasing enzyme, aminopeptidases, carboxypeptidases, and pyroglutamate aminopeptidase.

43. The method of claim 1, wherein the colloidal sol particle size is from about 1 nm to about 30 nm.

44. A method of preparing a microanalytical device, comprising forming a sol-gel comprising an entrapped biological molecule, crushing the sol-gel to particulates having a diameter of from about 10 82 m to about 80 μm, and forming the sol-gel particulates into a bed within the microanalytical device or on the surface of the microanalytical device.

45. A method of preparing a microanalytical device comprising forming a sol-gel comprising an entrapped biological molecule, wherein the form of said sol-gel is selected from the group consisting of a monolithic gel, thin film, or fiber and wherein the sol-gel is placed in or on the microanalytical device.

46. A method of using a microanalytical device comprising a sol-gel comprising an entrapped biological molecule, comprising applying an analyte sample to the bed, optionally applying additional buffer solution to the bed, and analyzing the eluant from the bed.

47. The method of claim 44 or 45, wherein the bed on the microanalytical device is in the form of a microcolumn or microchannel.

48. The method of claim 44 or 45, wherein the bed on the microanalytical device is in the form of a microarray.

49. The method of claim 46, wherein the eluant is analyzed using mass spectrometry.

50. The method of claim 46, wherein the eluant is analyzed using micro or capillary electrophoresis.

51. The method of claim 46, wherein the interaction of any component in the sample with the entrapped biological molecule in the sol-gel is measured using a method selected from the group consisting of UV/Visible, Near IR, fluorescence, refractive index (RI) and Raman spectroscopies.

52. The method of claim 46, further comprising washing the sol-gel with a solution to elute analytes from the sol-gel, and analyzing the analytes.

53. The method of claim 52, wherein the analytes are analyzed using mass spectrometry.

54. The method of claim 52, wherein the analytes are analyzed using a method selected from the group consisting of UV/Visible, Near IR, fluorescence, refractive index (RI) and Raman spectroscopies.

55. The method of claims 44 or 45, wherein the microanalytical device is fabricated by a method selected from the group consisting of silicon micromachining, microlithography, molding and etching.

56. The method of claim 45, wherein the sol-gel is formed in situ on the microanalytical device.

57. In a microanalytical device comprised of a substrate and at least one feature selected from microchannels, microcolumns, and combinations thereof, the improvement which comprises incorporating into said at least one feature particulates of sol-gel having a diameter of from about 10 μm to about 80 μm.

58. In a microanalytical device comprised of a substrate and at least one feature selected from microchannels, microcolumns, and combinations thereof, the improvement which comprises incorporating into said at least one feature and/or onto a surface of the substrate a sol-gel having a biological molecule entrapped therein, wherein the sol-gel is in a form selected from the group consisting of a monolithic gel, a thin film, and a fiber.

59. The microanalytical device of claims 57 or 58, adapted for performing high throughput screening of samples.
